(19) United States
(12) Patent Application Publication
(10) Pub. No.: US 2014/0316239 A1
(43) Pub. Date: Oct. 23, 2014

(54) LASER LIGHT SOURCE UNIT, METHOD OF CONTROLLING THE SAME, AND PHOTOACOUSTIC IMAGE GENERATION DEVICE

(71) Applicant: FUJIFILM CORPORATION, Tokyo (JP)

(72) Inventor: Tadashi KASAMATSU, Ashigarakami-gun (JP)

(21) Appl. No.: 14/321,085

(22) Filed: Jul. 1, 2014

Related U.S. Application Data
(63) Continuation of application No. PCT/JP2013/053371, filed on Feb. 13, 2013.

(30) Foreign Application Priority Data
Feb. 29, 2012 (JP) 2012-043270
Jan. 23, 2013 (JP) 2013-009774
Feb. 5, 2013 (JP) 2013-020074

(51) Int. Cl. H01S 3/121; A61B 5/00; H01S 3/08

(52) U.S. Cl. CPC H01S 3/121; H01S 3/08086; A61B 5/0095
USPC 600/407; 372/14

(57) ABSTRACT

Disclosed is a laser light source unit capable of emitting pulse laser light with desired emission intensity even at different wavelengths. When emitting pulse laser light having a wavelength of 750 nm, excitation energy of a flash lamp decreases compared to when emitting pulse laser light having a wavelength of 800 nm. Specifically, the charging time of a capacitor of a pulse generation circuit decreases compared to when emitting pulse laser light having a wavelength of 800 nm.

### CROSS-REFERENCE TO RELATED APPLICATIONS

This application is a Continuation of PCT International Application No. PCT/JP2013/053371 filed on Feb. 13, 2013, which claims priority under 35 U.S.C §119(a) to Japanese Patent Application No. 2012-043270 filed Feb. 29, 2012, Japanese Patent Application No. 2013-009774 filed Jan. 23, 2013 and Japanese Patent Application No. 2013-020074 filed Feb. 5, 2013. Each of the above application(s) is hereby expressly incorporated by reference, in its entirety, into the present application.

### BACKGROUND OF THE INVENTION

1. Field of the Invention

The present invention relates to a laser light source unit and a method of controlling the same, and in particular, to a laser light source unit capable of switching and emitting laser light having a plurality of wavelengths and a method of controlling the same.

The present invention relates to a photoacoustic image generation device, and in particular, to a photoacoustic image generation device which irradiates laser light having a plurality of wavelengths onto a subject to detect a photoacoustic signal and generates a photoacoustic image based on the detected photoacoustic signal.

2. Description of the Related Art

In the related art, for example, as shown in JP2005-21380A or A High-Speed Photoacoustic Tomography System based on a Commercial Ultrasound and a Custom Transducer Array, Xueding Wang, Jonathan Cannata, Derek DeBusschere, Changhong Hu, J. Brian Fowlkes, and Paul Carson, Proc. SPIE Vol. 7564, 756424 (Feb. 23, 2010), a photoacoustic imaging device which images the inside of a living body using a photoacoustic effect is known. In this photoacoustic imaging device, for example, pulse light, such as pulse laser light, is irradiated onto the living body. Inside the living body onto which pulse light is irradiated, a tissue of the living body which absorbs energy of pulse light expands in volume due to heat, and an acoustic wave is generated. The acoustic wave is detected by the ultrasound probe or the like, and the inside of the living body can be visualized based on the detected signal (photoacoustic signal). In a photoacoustic imaging method, since an acoustic wave is generated in a specific optical absorber, a specific tissue in the living body, for example, a blood vessel or the like can be imaged.

On the other hand, a large number of living body tissues have an optical absorption characteristic which changes depending on the wavelength of light, and in general, the optical absorption characteristic is peculiar to each tissue. For example, FIG. 10 shows molecular absorption coefficients for each light wavelength of oxygenated hemoglobin (hemoglobin bonded to oxygen: oxy-Hb) contained in a large amount in a human artery and deoxygenated hemoglobin (hemoglobin not bonded to oxygen: deoxy-Hb) contained in a large amount in a vein. The optical absorption characteristic of the artery corresponds to the optical absorption characteristic of oxygenated hemoglobin, and the optical absorption characteristic of the vein corresponds to the optical absorption characteristic of deoxygenated hemoglobin. A photoacoustic imaging method which irradiates light having two different wavelengths onto a blood vessel portion by means of a difference in light absorption depending on wavelength and examines the magnitude of a photoacoustic signal obtained at each wavelength to distinguish between a photoacoustic signal from an artery and a photoacoustic signal from a vein, and images the artery and the vein distinctively is known (see JP2010-046215A).

In a laser light source, since an oscillation gain is different depending on wavelength, emission intensity is different. For this reason, in a wavelength sweeping gas laser device, a method in which a laser gain, specifically, an excitation current or the like is controlled to maintain laser emission intensity constant regardless of change in wavelength is suggested (see JP1997-114292A (JP-S52-114292A)).

In the photoacoustic imaging method, as described above, although light having two wavelengths is irradiated onto the blood vessel portion of the subject and the artery and the vein are imaged distinctively, if the emission intensity of laser light is different depending on wavelength, the magnitude of a photoacoustic signal to be detected is different depending on wavelength, and as a result, there is a concern that it is difficult to distinguish between the artery and the vein. The method described in JP1997-114292A is suitable for a gas laser, not a solid-state laser.

### SUMMARY OF THE INVENTION

The invention has been accomplished in consideration of the above-described situation, and an object of the invention is to provide a laser light source unit capable of emitting pulse laser light with desired emission intensity even at different wavelengths and a method of controlling the same. An object of the invention is to provide a photoacoustic image generation device including the laser light source unit.

According to an aspect of the invention, there is provided a laser light source unit which sequentially emits a plurality of beams of pulse laser light in a predetermined wavelength sequence including two or more different wavelengths. The laser light source unit includes a laser rod, an excitation light source which irradiates excitation light onto the laser rod, an optical resonator which includes a pair of mirrors facing each other with the laser rod interposed therebetween, a laser light emission part for emitting pulse laser light, a wavelength switching part which is inserted inside the optical resonator and changes the oscillation wavelength of the optical resonator, an excitation light control part for controlling excitation energy supplied to the excitation light source according to the wavelength of pulse laser light to be emitted and irradiating the excitation light from the excitation light source onto the laser rod, and an emission control part for switching the oscillation wavelength by the wavelength switching part to adjust the wavelength of pulse laser light to be emitted in synchronization with the irradiation of excitation light from the excitation light source onto the laser rod and causing the laser light emission part to emit pulse laser light.

In the laser light source unit according to the aspect of the invention, the excitation light control part may be a part for increasing excitation energy as a wavelength or the emission intensity of pulse laser light from the laser rod is weak when excitation is performed with a uniform amount of light.

In the laser light source unit according to the aspect of the invention, the predetermined wavelength sequence may include a first wavelength and a second wavelength, and when the emission intensity of pulse laser light having the second wavelength is weaker than the emission intensity of pulse laser light having the first wavelength, the excitation light control part may be a part for supplying first excitation energy to the excitation light source when the oscillation wavelength is the first wavelength and supplying second excitation energy greater than the first excitation energy to the excitation light source when the oscillation wavelength is the second wavelength.

In the laser light source unit according to the aspect of the invention, the laser rod may be alexandrite, the first wavelength may be 748 nm to 770 nm, the second wavelength may be 793 nm to 802 nm, and the second excitation energy may have a value 1.8 to 2.2 times the first excitation energy.

In the laser light source unit according to the aspect of the invention, the excitation light control part may be a part including a capacitor, which is charged to cause the excitation light to be emitted from the excitation light source, for controlling the charging time of the capacitor according to the wavelength to control excitation energy supplied to the excitation light source.

In the laser light source unit according to the aspect of the invention, the excitation light control part may be a part including a capacitor, which is charged to cause the excitation light to be emitted from the excitation light source, for controlling the charging time of the capacitor according to the wavelength to control excitation energy supplied to the excitation light source.

In the laser light source unit according to the aspect of the invention, the laser light emission part may be a Q switch which is inserted inside the optical resonator.

According to another aspect of the invention, there is provided a photoacoustic image generation device including a laser light source unit which sequentially emits a plurality of beams of pulse laser light in a predetermined wavelength sequence including two or more different wavelengths, a detection part for detecting a photoacoustic signal generated in a subject when pulse laser light having each wavelength in the predetermined wavelength sequence is irradiated onto the subject and generating photoacoustic data corresponding to each wavelength, an intensity ratio extraction part for extracting the magnitude relationship of relative signal intensity between photoacoustic data corresponding to the respective wavelengths, and a photoacoustic image construction part for generating a photoacoustic image based on the extracted magnitude relationship. The laser light source unit includes a laser rod, an excitation light source which irradiates excitation light onto the laser rod, an optical resonator which includes a pair of mirrors facing each other with the laser rod interposed therebetween, a laser light emission part for emitting pulse laser light, a wavelength switching part which is inserted inside the optical resonator and changes the oscillation wavelength of the optical resonator, an excitation light control part for controlling excitation energy supplied to the excitation light source according to the wavelength of pulse laser light to be emitted and irradiating the excitation light from the excitation light source onto the laser rod, and an emission control part for switching the oscillation wavelength by the wavelength switching part to adjust the wavelength of pulse laser light to be emitted in synchronization with the irradiation of excitation light from the excitation light source onto the laser rod and causing the laser light emission part to emit pulse laser light.

The photoacoustic image generation device according to the aspect of the invention may further include an intensity information extraction part for generating intensity information representing signal intensity based on photoacoustic data corresponding to each wavelength, in which the photoacoustic image construction part may be a part for determining the gradation value of each pixel of the photoacoustic image based on the intensity information and determining the display color of each pixel based on the extracted magnitude relationship.

In the photoacoustic image generation device according to the aspect of the invention, the detection part may be a part for detecting a reflected ultrasonic wave to an ultrasonic wave transmitted to the subject to generate reflected ultrasonic data, and the photoacoustic image generation device may further include an ultrasound image generation part for generating an ultrasound image based on the reflected ultrasonic data.

According to still another aspect of the invention, there is provided a method of controlling a laser light source unit, which sequentially emits a plurality of beams of pulse laser light in a predetermined wavelength sequence including two or more different wavelengths, including a step of controlling excitation energy supplied to an excitation light source, which irradiates excitation light onto a laser rod, among two or more excitation light sources, which irradiate excitation light onto the laser rod, according to the wavelength of pulse laser light to be emitted, a step of irradiating excitation light from the controlled excitation light source onto the laser rod, a step of switching an oscillation wavelength by a wavelength switching part, which is inserted inside an optical resonator including a pair of mirrors facing each other with the laser rod interposed therebetween and changes the oscillation wavelength of the optical resonator, to adjust the wavelength of pulse laser light to be emitted in synchronization with the irradiation of excitation light from the excitation light source onto the laser rod, and a step of causing a laser light emission part for emitting pulse laser light to emit pulse laser light.

According to the invention, excitation energy supplied to the excitation light source is controlled according to the wavelength of pulse laser light to be emitted to irradiate excitation light from the excitation light source onto the laser rod, the oscillation wavelength is switched by the wavelength switching part to adjust the wavelength of pulse laser light to be emitted in synchronization with the irradiation of excitation light, and pulse laser light is emitted by the laser light emission part. For this reason, excitation energy increases as the emission intensity of pulse laser light is weak when excitation is performed with a uniform amount of light, whereby it is possible to make the emission intensity of pulse laser light uniform regardless of the wavelength of pulse laser light to be emitted. Since it is possible to make the emission intensity of pulse laser light uniform only by controlling excitation energy supplied to the excitation light source, the configuration of the laser light source unit can be made simple and inexpensive.

As described above, the photoacoustic signal generated in the subject when pulse laser light having each wavelength with uniform emission intensity is irradiated onto the subject is detected, photoacoustic data corresponding to each wavelength is generated, the magnitude relationship of relative signal intensity between photoacoustic data corresponding to the respective wavelengths is extracted, and the photoacoustic image is generated based on the extracted magnitude relationship. Accordingly, since it is possible to accurately extract the magnitude relationship of relative signal intensity between photoacoustic data, it is possible to generate a photoacoustic image suitable for diagnosis.

### BRIEF DESCRIPTION OF THE DRAWINGS

FIG. 1 is a block diagram showing the configuration of a photoacoustic image generation device to which a laser light source unit according to a first embodiment of the invention is applied.

FIG. 2 is a block diagram showing the configuration of the laser light source unit according to the first embodiment.

FIG. 3 is a diagram showing the configuration of a flash lamp power supply of the laser light source unit according to the first embodiment.

FIG. 4 is a graph showing the relationship between the wavelength of laser light emitted from alexandrite crystal and output energy at 60 degrees Celsius.

FIG. 5 is a perspective view showing a configuration example of a wavelength switching part, a drive part, and a drive state detection part.

FIG. 6 is a flowchart (first view) showing processing which is performed in the first embodiment.

FIG. 7 is a flowchart (second view) showing processing which is performed in the first embodiment.

FIG. 8 is a diagram showing the configuration of a flash lamp power supply of a laser light source unit according to a second embodiment.

FIG. 9 is a flowchart showing processing which is performed in the second embodiment.

FIG. 10 is a graph showing molecular absorption coefficients for each light wavelength of oxygenated hemoglobin and deoxygenated hemoglobin.

### DESCRIPTION OF THE PREFERRED EMBODIMENTS

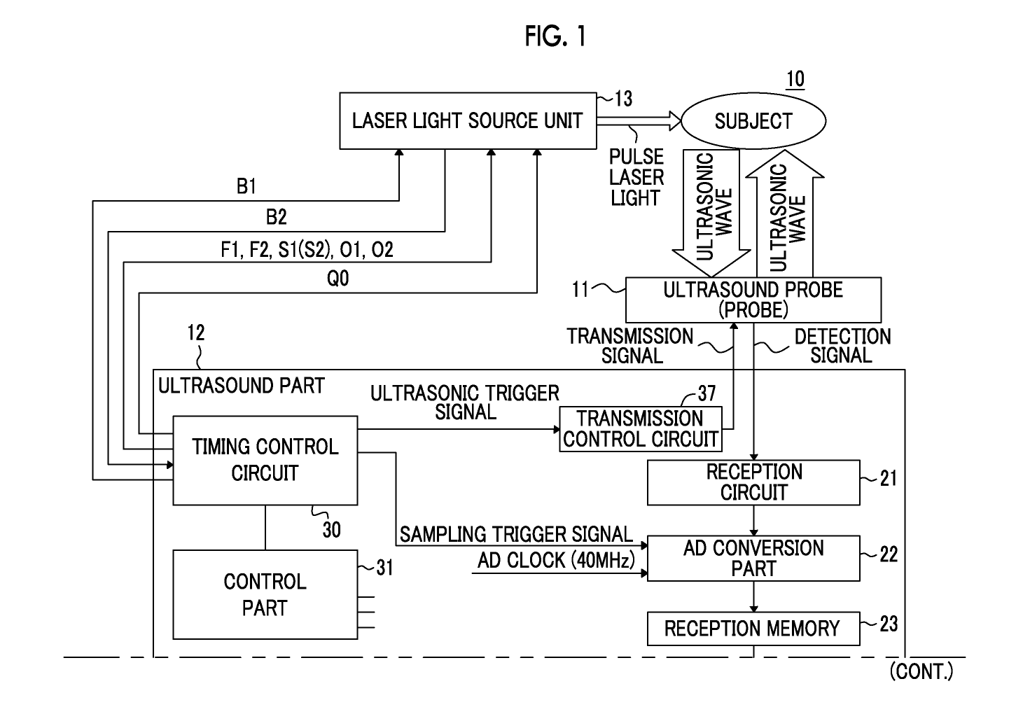

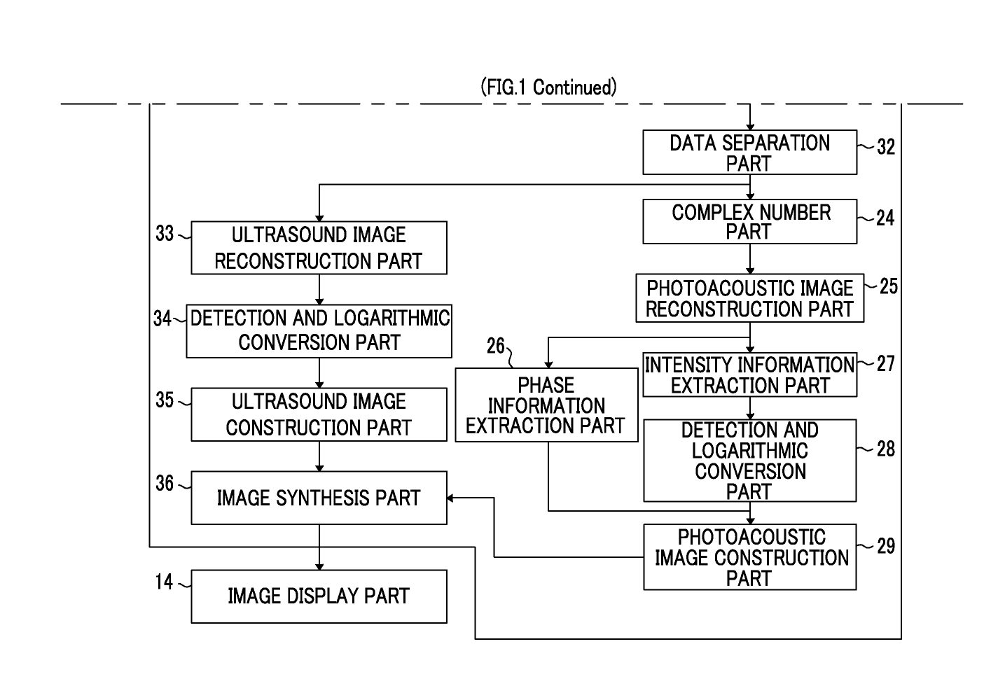

Hereinafter, an embodiment of the invention will be described in detail referring to the drawings. FIG. 1 is a block diagram showing the configuration of a photoacoustic image generation device to which a laser light source unit according to a first embodiment of the invention is applied. As shown in FIG. 1, a photoacoustic image generation device 10 includes an ultrasound probe (probe) 11, an ultrasound part 12, and a laser light source unit 13. The laser light source unit 13 emits pulse laser light to be irradiated onto a subject. The laser light source unit 13 switches and emits pulse laser light having a plurality of different wavelengths. In the following description, a case where the laser light source unit 13 sequentially emits pulse laser light having a first wavelength and pulse laser light having a second wavelength will be primarily described.

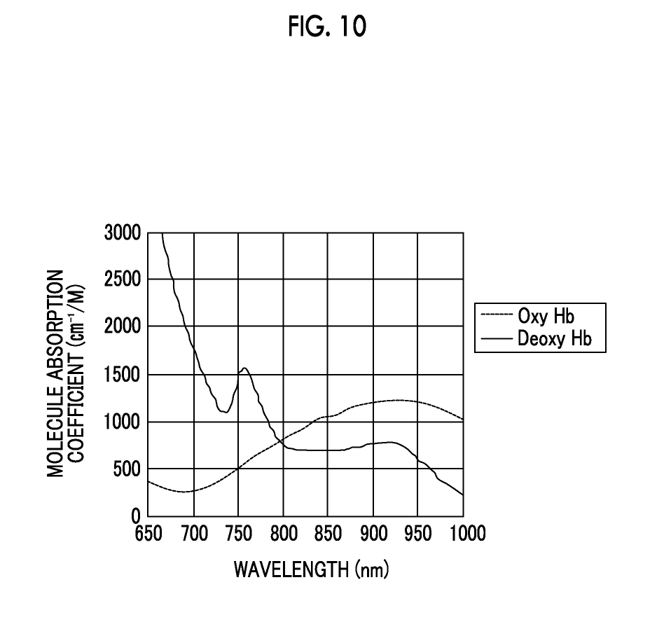

In this embodiment, for example, the first wavelength (center wavelength) of about 750 nm is considered, and the second wavelength of about 800 nm is considered. In this case, the first wavelength (about 750 nm) and the second wavelength (about 800 nm) are in a predetermined wavelength sequence. Referring to FIG. 10 described above, a molecular absorption coefficient at a wavelength of 750 nm of oxygenated hemoglobin (hemoglobin bonded to oxygen: oxy-Hb) contained in a large amount in a human artery is lower than a molecular absorption coefficient at a wavelength of 800 nm. A molecular absorption coefficient at a wavelength of 750 nm of deoxygenated hemoglobin (hemoglobin not bonded to oxygen: deoxy-Hb) contained in a large amount in a vein is higher than a molecular absorption coefficient at a wavelength of 800 nm. This nature is use to check whether a photoacoustic signal obtained at the wavelength of 750 nm is relatively greater or smaller than a photoacoustic signal obtained at the wavelength of 800 nm, thereby distinguishing between the photoacoustic signal from the artery and the photoacoustic signal from the vein.

For example, pulse laser light emitted from the laser light source unit 13 is guided to the probe 11 by a part of a light guide, such as an optical fiber, and is irradiated from the probe 11 onto the subject. The irradiation position of pulse laser light is not particularly limited, and pulse laser light may be irradiated from a place other than the probe 11. In the subject, an optical absorber absorbs energy of irradiated pulse laser light, whereby an ultrasonic wave (acoustic wave) is generated. The probe 11 includes an ultrasonic detector. For example, the probe 11 has a plurality of ultrasonic detector elements (ultrasound transducer) arranged in a one-dimensional manner, and detects an acoustic wave (photoacoustic signal) from the subject by the ultrasound transducer arranged in a one-dimensional manner. In this embodiment, the probe 11 performs the output (transmission) of an ultrasonic wave to the subject and the detection (reception) of a reflected ultrasonic wave from the subject for the transmitted ultrasonic wave, in addition to the detection of the photoacoustic signal.

The ultrasound part 12 has a reception circuit 21, an AD conversion part 22, a reception memory 23, a complex number part 24, a photoacoustic image reconstruction part 25, a phase information extraction part 26, an intensity information extraction part 27, a detection and logarithmic conversion part 28, a photoacoustic image construction part 29, a timing control circuit 30 (a light emission control part), and a control part 31. The ultrasound part 12 also has, for generating an ultrasound image, a data separation part 32, an ultrasound image reconstruction part 33, a detection and logarithmic conversion part 34, an ultrasound image construction part 35, an image synthesis part 36, and a transmission control circuit 37.

The reception circuit 21 receives the photoacoustic signal and the reflected ultrasonic signal detected by the probe 11. The AD conversion part 22 is a detection part, samples the photoacoustic signal and the reflected ultrasonic signal received by the reception circuit 21, and generates photoacoustic data and reflected ultrasonic data which are digital data. The AD conversion part 22 receives a sampling trigger signal transmitted from the timing control circuit 30 in conformity with the timing of the ultrasonic transmission and starts sampling of the reflected ultrasonic wave. The AD conversion part 22 performs sampling of the photoacoustic signal and the reflected ultrasonic signal in a predetermined sampling period in synchronization with an AD clock signal.

The AD conversion part 22 stores photoacoustic data corresponding to each wavelength of pulse laser light emitted from the laser light source unit 13 in the reception memory 23. That is, the AD conversion part 22 stores, in the reception memory 23, first photoacoustic data obtained by sampling the photoacoustic signal detected by the probe 11 when pulse laser light having the first wavelength is irradiated onto the subject and second photoacoustic data obtained by sampling the photoacoustic signal detected by the probe 11 when pulse laser light having the second wavelength is irradiated. The AD conversion part 22 also stores, in the reception memory 23, reflected ultrasonic data obtained by sampling the reflected ultrasonic signal detected by the probe 11 when an ultrasonic wave is transmitted to the subject.

The complex number part 24 reads the first photoacoustic data and the second photoacoustic data from the reception memory 23, and generates complex data in which one of the first photoacoustic data and the second photoacoustic data is a real part and the other data is an imaginary part. Hereinafter, a case where the complex number part 24 generates complex data in which the first photoacoustic data is a real part and the second photoacoustic data is an imaginary part will be described.

The photoacoustic image reconstruction part 25 receives complex data from the complex number part 24 as input. The photoacoustic image reconstruction part 25 performs image reconstruction from input complex data by a Fourier transformation method (FTA method). In the image reconstruction by the Fourier transformation method, for example, a known method in the related art described in “Photoacoustic Image Reconstruction-A Quantitative Analysis” Jonathan I. Sperl et al. SPIE-OSA Vol. 6631 663103 or the like can be applied. The photoacoustic image reconstruction part 25 inputs Fourier transformed data representing a reconstructed image to the phase information extraction part 26 and the intensity information extraction part 27.

The phase information extraction part 26 extracts the magnitude relationship of relative signal intensity between photoacoustic data corresponding to the respective wavelengths. In this embodiment, the phase information extraction part 26 has a reconstructed image reconstructed by the photoacoustic image reconstruction part 25 as input data, and extracts phase information, which represents how much one of the real part and the imaginary part is greater than the other part, from input data as complex data. For example, when complex data is expressed by X+iY, the phase information extraction part 26 generates θ=tan⁻¹(Y/X) as the phase information. When X=0, θ=90°. When the first photoacoustic data (X) constituting the real part and the second photoacoustic data (Y) constituting the imaginary part are equal to each other, the phase information becomes θ=45°. The phase information becomes close to θ=0° as the first photoacoustic data is relatively greater, and becomes close to θ=90° as the second photoacoustic data is greater.

The intensity information extraction part 27 generates intensity information representing signal intensity based on photoacoustic data corresponding to each wavelength. In this embodiment, the intensity information extraction part 27 has a reconstructed image reconstructed by the photoacoustic image reconstruction part 25 as input data, and generates intensity information from input data as complex data. For example, when complex data is expressed by X+iY, the intensity information extraction part 27 extracts (X²+Y²)^{1/2} as the intensity information. The detection and logarithmic conversion part 28 generates an envelope of data representing the intensity information extracted by the intensity information extraction part 27 and logarithmically converts the envelope to expand a dynamic range.

The photoacoustic image construction part 29 receives the phase information from the phase information extraction part 26 as input and receives the intensity information after the detection and logarithmic conversion processing from the detection and logarithmic conversion part 28 as input. The photoacoustic image construction part 29 generates a photoacoustic image, which is a distribution image of an optical absorber, based on the input phase information and intensity information. The photoacoustic image construction part 29 determines the luminance (gradation value) of each pixel in the distribution image of the optical absorber based on the input intensity information. The photoacoustic image construction part 29 determines the color (display color) of each pixel in the distribution image of the optical absorber based on the phase information. The photoacoustic image construction part 29 determines the color of each pixel based on the input phase information by means of a color map, in which a phase range of 0° to 90° corresponds to predetermined colors.

Since a phase range of 0° to 45° is a range in which the second photoacoustic data is greater than the first photoacoustic data, it is considered that the generation source of the photoacoustic signal is a vein in which blood primarily containing deoxygenated hemoglobin having greater absorption for the wavelength of 750 nm than absorption for the wavelength of 800 nm flows. Since a phase range of 45° to 90° is a range in which the second photoacoustic data is smaller than the first photoacoustic data, it is considered that the generation source of the photoacoustic signal is an artery in which blood primarily containing oxygenated hemoglobin having smaller absorption for the wavelength of 750 nm than absorption for the wavelength of 800 nm flows.

Accordingly, as the color map, for example, a color map in which the phase 0° is blue, color gradually changes to be colorless (white) as the phase becomes close to 45°, the phase 90° is red, and color gradually changes to be white as the phase becomes close to 45° is used. In this case, on the photoacoustic image, a portion corresponding to an artery can be expressed in red, and a portion corresponding to a vein can be expressed in blue. The gradation value may be constant, and color coding of the portion corresponding to the artery and the portion corresponding to the vein may be merely performed according to the phase information without using the intensity information. An image display part 14 displays the photoacoustic image generated by the photoacoustic image construction part 29 on a display screen along with an ultrasound image described below.

The data separation part 32 separates the reflected ultrasonic data stored in the reception memory 23 from the first and second photoacoustic data, inputs the reflected ultrasonic data to the ultrasound image reconstruction part 33, and inputs the first and second photoacoustic data to the complex number part 24.

The ultrasound image reconstruction part 33 generates data of each line of an ultrasound image based on (the sampling data of) the reflected ultrasonic wave detected by a plurality of ultrasound transducers of the probe 11. For example, the ultrasound image reconstruction part 33 adds data from 64 ultrasound transducers of the probe 11 with a delay time according to the position of each ultrasound transducer to generate data for one line (delay addition method).

The detection and logarithmic conversion part 34 obtains an envelope of data of each line output from the ultrasound image reconstruction part 33 and performs logarithmic conversion for the obtained envelope. The ultrasound image construction part 35 generates an ultrasound image based on data of each line subjected to logarithmic conversion. The ultrasound image reconstruction part 33, the detection and logarithmic conversion part 34, and the ultrasound image construction part 35 configure an ultrasound image generation part for generating the ultrasound image based on the reflected ultrasonic wave.

The image synthesis part 36 synthesizes the photoacoustic image and the ultrasound image. For example, the image synthesis part 36 performs image synthesis by superimposing the photoacoustic image and the ultrasound image. At this time, it is preferable that the image synthesis part 36 performs positioning such that the corresponding points are the same positions of the photoacoustic image and the ultrasound image. A synthesized image is displayed on the image display part 14. Image synthesis may not be performed, and the photoacoustic image and the ultrasound image may be displayed in parallel on the image display part 14, or the photoacoustic image and the ultrasound image may be switched.

If an ultrasonic transmission trigger signal which is transmitted from the timing control circuit 30 at the time of the generation of the ultrasound image and instructs ultrasonic transmission is received, the transmission control circuit 37 causes an ultrasonic wave to be transmitted from the probe 11. After the transmission of the ultrasonic wave, the probe 11 detects a reflected ultrasonic wave from the subject.

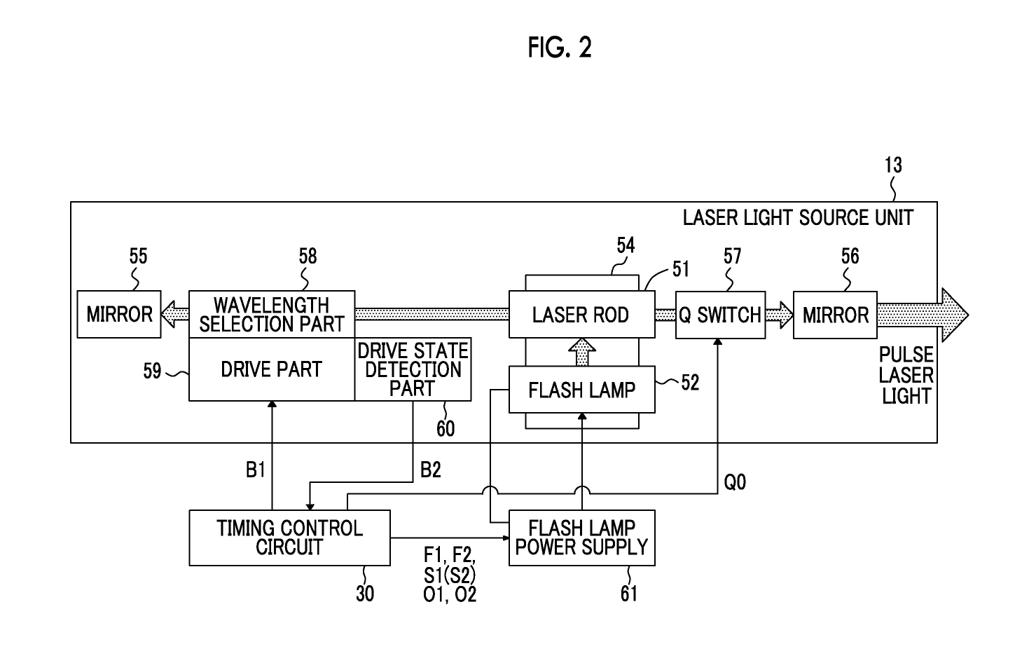

Next, the configuration of the laser light source unit 13 will be described in detail. FIG. 2 is a block diagram showing the configuration of the laser light source unit 13 according to the first embodiment. As shown in FIG. 2, the laser light source unit 13 includes a laser rod 51, a flash lamp 52, an excitation chamber 54, mirrors 55 and 56, a Q switch 57, a wavelength switching part 58, a drive part 59, and a drive state detection part 60.

The laser rod 51 is a laser medium. For the laser rod 51, for example, alexandrite crystal, Cr:LiSAF (Cr:LiSrAlF₆) crystal, Cr:LiCAF (Cr:LiCaAlF₆) crystal, or Ti:Sapphire crystal may be used. In this embodiment, it is assumed that alexandrite crystal is used.

The flash lamp 52 is an excitation light source, and irradiates excitation light onto the laser rod 51. For the flash lamp 52, for example, a Xenon lamp may be used. The flash lamp 52 is controlled to be turned on by a flash lamp power supply 61 described below.

The excitation chamber 54 accommodates the laser rod 51 and the flash lamp 52. In the excitation chamber 54, cooling water circulates and cools the laser rod 51 and the flash lamp 52.

The mirrors 55 and 56 face each other with the laser rod 51 interposed therebetween, and an optical resonator is configured by the mirrors 55 and 56. It is assumed that the mirror 56 is on an output side. In the optical resonator, the Q switch 57 (a laser light emission part) is inserted. Insertion loss in the optical resonator is rapidly changed from great loss (low Q) to small loss (high Q) by the Q switch 57, thereby obtaining pulse laser light. Instead of the Q switch in the optical resonator, a part, such as a chopper, for cutting a short pulse may be provided outside the optical resonator.

The wavelength switching part 58 includes a plurality of bandpass filters (BPFs) which have different transmission wavelengths. The wavelength switching part 58 selectively inserts a plurality of bandpass filters on the optical path of the optical resonator. The wavelength switching part 58 includes a first bandpass filter which transmits light having a wavelength of 750 nm (center wavelength), and a second bandpass filter which transmits light having a wavelength of 800 nm (center wavelength). The first bandpass filter is inserted on the optical path of the optical resonator, thereby setting the oscillation wavelength of an optical oscillator to 750 nm, and the second bandpass filter is inserted on the optical path of the optical resonator, thereby setting the oscillation wavelength of an optical oscillator to 800 nm.

The drive part 59 drives the wavelength switching part 58 such that a bandpass filter which is inserted on the optical path of the optical resonator is sequentially switched in a predetermined order. For example, when the wavelength switching part 58 is configured as a filter rotor which switches a bandpass filter to be selectively inserted on the optical path of the optical resonator with rotation displacement, the drive part 59 rotates and drives the filter rotor which configures the wavelength switching part 58. The drive state detection part 60 detects the drive state of the wavelength switching part 58. For example, the drive state detection part 60 detects the rotation displacement of the wavelength switching part 58 as the filter rotor. The drive state detection part 60 outputs a BPF state signal B2 representing the rotation displacement position of the filter rotor to the ultrasound part 12.

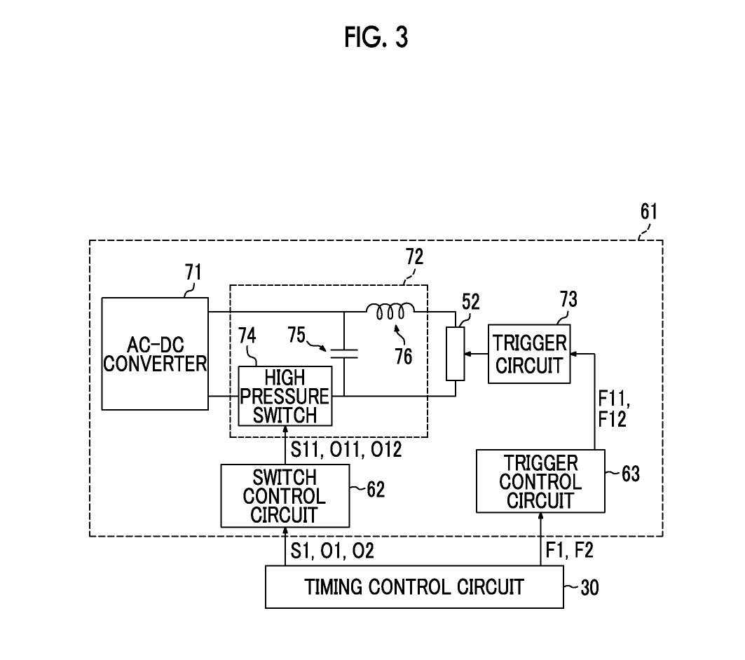

Next, the configuration of a flash lamp power supply will be described in detail. FIG. 3 is a diagram showing the configuration of a flash lamp power supply of the laser light source unit according to the first embodiment. A flash lamp power supply 61 is an excitation light control part, and includes an AC-DC converter 71, a pulse generation circuit (Pulse forming Network) 72, a trigger circuit 73, a switch control circuit 62, and a trigger control circuit 63.

The AC-DC converter 71 is a boosting circuit which boosts a voltage from an AC power supply (not shown) to a voltage necessary for causing the flash lamp 52 to emit light. The AC-DC converter 71 has a known circuit in which a diode bridge rectifier circuit and a boosting transformer are incorporated, and the output voltage of the AC-DC converter 71 is variable.

The pulse generation circuit 72 includes a high pressure switch 74, a capacitor 75 having predetermined capacitance, and a coil 76. The high pressure switch 74 is turned on according to a switch signal S11 from the switch control circuit 62 and charges the capacitor 75. If charging is made enough to cause the flash lamp 52 to emit light, flash lamp trigger signals (F/L trigger signals) F11 and F12 are output from the trigger control circuit 63 to the trigger circuit 73. While the flash lamp 52 is usually in an insulation state, the flash lamp 52 is placed in an electrical conduction state by the trigger circuit 73 which receives the flash lamp trigger signals F11 and F12, and accordingly, the flash lamp 52 is turned on. Instead of the high pressure switch, a switch may be provided on a low-voltage primary side (AC side), and the switch may be turned on, thereby charging the capacitor.

The switch control circuit 62 is a circuit which receives a switch signal S1 from the timing control circuit 30 and outputs the switch signal S11 for turning on the high pressure switch 74 to the high pressure switch 74. The trigger control circuit 63 is a circuit which receives flash lamp trigger signals F1 and F2 from the timing control circuit 30 and outputs the flash lamp trigger signals F11 and F12 for placing the flash lamp 52 in the electrical conduction state to the trigger circuit 73.

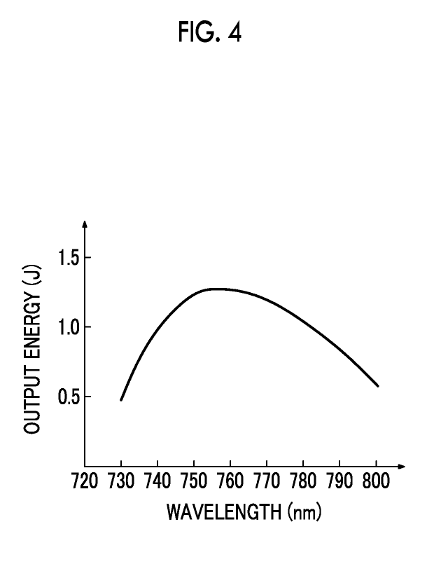

In this embodiment, although pulse laser light having the wavelengths of 750 nm and 800 nm is emitted to generate a photoacoustic image, when the laser rod 51 is excited with a uniform amount of light, the emission intensity of pulse laser light emitted from the laser light source unit 13 is different depending on wavelength. FIG. 4 is a graph showing the relationship between the wavelengths of laser light emitted from alexandrite crystal for use in the laser rod 51 and the output energy at 60 degrees Celsius. As shown in FIG. 4, the output energy (that is, emission intensity) at 750 nm is twice the output energy at 800 nm. The value of twice changes under a measurement condition, such as a measurement temperature and becomes a value 1.8 times to 2.2 times.

For this reason, in this embodiment, when emitting pulse laser light having the wavelength of 750 nm, the charging time of the capacitor 75 is set to ½ such that the excitation energy of the laser rod 51 becomes ½, compared to when emitting pulse laser light having the wavelength of 800 nm. Specifically, if the charging time when emitting pulse laser light having the wavelength of 800 nm is set to T0, when emitting pulse laser light having the wavelength of 750 nm, the charging time of the capacitor 75 is set to ½T0. With this, the amount of excitation light irradiated onto the laser rod 51 when emitting pulse laser light having the wavelength of 750 nm becomes ½ of the amount of light when emitting pulse laser light having the wavelength of 800 nm. Accordingly, it is possible to make the emission intensity of pulse laser light having the wavelength of 750 nm and the emission intensity of pulse laser light having the wavelength of 800 nm uniform.

The charging time ½T0 and T0 are controlled by switch off signals O1 and O2 output from the timing control circuit 30. That is, when emitting pulse laser light having the wavelength of 750 nm, the timing control circuit 30 outputs the switch off signal O1 at the timing of ½T0 after the charging of the capacitor 75 starts, and the switch control circuit 62 outputs a switch off signal O11 to the high pressure switch 74 in response to the switch off signal O1. With this, the high pressure switch 74 is turned off, and the charging of the capacitor 75 is completed. When emitting pulse laser light having the wavelength of 800 nm, the timing control circuit 30 outputs the switch off signal O2 at the timing of T0 after the charging of the capacitor 75 starts, and the switch control circuit 62 outputs a switch off signal O12 to the high pressure switch 74 in response to the switch off signal O2. With this, the high pressure switch 74 is turned off, and the charging of the capacitor 75 is completed.

A system in which the capacitance C of the capacitor 75=100 μF, the inductance L of the coil 76=100 μH, and a pulse width=3√LC=300 μsec is considered. If a charging voltage is 2000 V, excitation energy at the time of full charging becomes ½CV²=200 J. If the charging time of the capacitor 75 at this time is T0, when the output P of the power supply=1500 W, T0=½CV²/P=133 msec. Accordingly, ½T0=67 msec.

Returning to FIG. 1, the control part 31 performs control of the respective parts in the ultrasound part 12. The timing control circuit 30 controls the drive part 59 such that a bandpass filter which is inserted on the optical path of the optical resonator by the wavelength switching part 58 in the laser light source unit 13 is switched at a predetermined switching speed. For example, the timing control circuit 30 controls the drive part 59 such that the filter rotor which configures the wavelength switching part 58 continuously rotates in a predetermined direction at a predetermined rotation speed. The rotation speed of the filter rotor can be, for example, determined based on the number of wavelengths (the number of transmission wavelengths of the bandpass filter) of pulse laser light emitted from the laser light source unit 13 and the number of emissions of pulse laser light per part time.

The timing control circuit 30 outputs a BPF control signal B1 for controlling the driving of the wavelength switching part 58. The drive part 59 of the laser light source unit 13 drives the wavelength switching part 58 according to the BPF control signal B1. The timing control circuit 30 controls the drive part 59 through the BPF control signal B1 such that the amount of change of a BPF state signal becomes the amount of change according to a predetermined switching speed (the rotation speed of the filter rotor) of the bandpass filter for a predetermined time.

In addition to the above, the timing control circuit 30 outputs the switch signal S1 for turning on the high pressure switch 74 and a flash lamp trigger signal F1 for controlling the light emission of the flash lamp 52 to the laser light source unit 13, and causes excitation light to be irradiated from the flash lamp 52 onto the laser rod 51. The timing control circuit 30 outputs the switch signal S1 and the flash lamp trigger signal F1 based on the BPF state signal B2. For example, the timing control circuit 30 outputs switch signal S1 if the BPF state signal B2 represents a state which shows the drive position of the wavelength switching part 58 in which a bandpass filter corresponding to the wavelength of pulse laser light to be emitted is inserted on the optical path of the optical resonator, and outputs the flash lamp trigger signal F1 and causes excitation light to be irradiated onto the laser rod 51 if the BPF state signal B2 becomes information which shows a position determined by subtracting the amount of displacement of the wavelength switching part 58 from the drive position for the time necessary for excitation of the laser rod 51.

The timing control circuit 30 outputs a Q switch trigger signal Q0 to the Q switch 57 at the timing, at which the wavelength switching part 58 inserts a bandpass filter having a transmission wavelength corresponding to the wavelength of pulse laser light to be emitted on the optical path of the optical resonator, after the irradiation of the excitation light. For example, when the wavelength switching part 58 is configured as a filter rotor, the timing control circuit 30 outputs the Q switch trigger signal Q0 when the BPF state signal B2 represents a position which shows the insertion of a bandpass filter corresponding to the wavelength of pulse laser light to be emitted on the optical path of the optical resonator. The Q switch 57 rapidly changes insertion loss in the optical resonator from great loss to small loss (the Q switch is turned on) in response to the Q switch trigger signal Q0, whereby pulse laser light is emitted from the output-side mirror 56.

The timing control circuit 30 outputs a sampling trigger signal (AD trigger signal) to the AD conversion part 22 in conformity with the timing of the Q switch trigger signal Q0, that is, the emission timing of pulse laser light. The AD conversion part 22 starts sampling of the photoacoustic signal based on the sampling trigger signal. The timing control circuit 30 outputs an ultrasonic trigger signal to the transmission control circuit 37 and outputs the sampling trigger signal to the AD conversion part 22 in conformity with the timing of the generation of the ultrasound image. The transmission control circuit 37 causes an ultrasonic wave to be transmitted from the probe 11 based on the ultrasonic trigger signal. The AD conversion part 22 starts sampling of the reflected ultrasonic signal based on the sampling trigger signal.

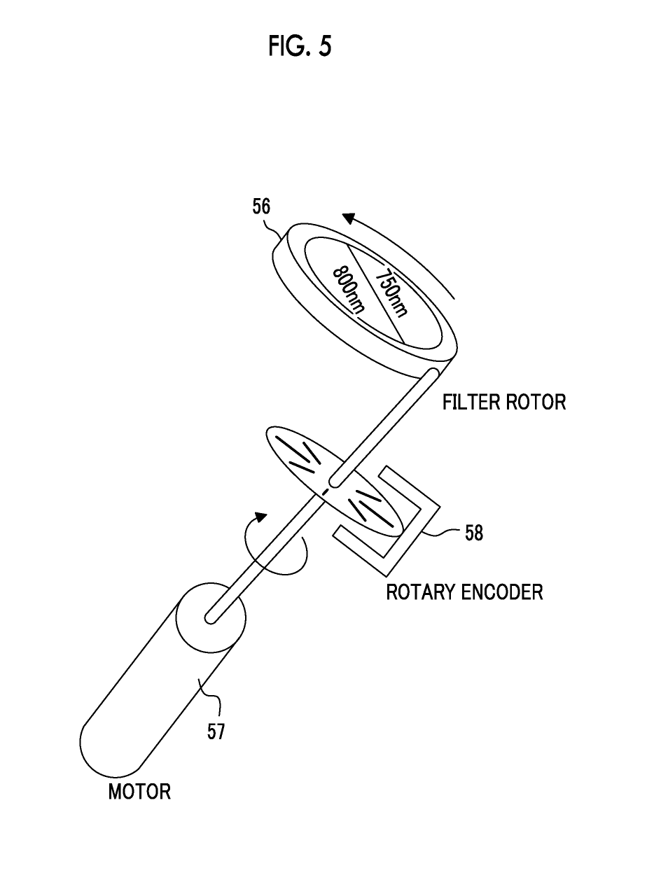

FIG. 5 is a perspective view showing a configuration example of the wavelength switching part 58, the drive part 59, and the drive state detection part 60. In this example, the wavelength switching part 58 is a filter rotor including two bandpass filters, and the drive part 59 is a servo motor. The drive state detection part 60 is a rotary encoder. The wavelength switching part 58 rotates with the rotation of the output shaft of the servo motor. Half (for example, from the rotation displacement position 0° to 180° the filter rotor which configures wavelength switching part 58 is a first bandpass filter which transmits light having the wavelength of 750 nm, and the other half (for example, from the rotation displacement position 180° to 360° is a second bandpass filter which transmits light having the wavelength of 800 nm. The filter rotor is rotated, whereby the first bandpass filter and the second bandpass filter can be alternately switched at a switching speed according to the rotation speed of the filter rotor and inserted on the optical path of the optical resonator.

The rotary encoder detects the rotation displacement of the filter rotor by a slitted rotary plate attached to the output shaft of the servo motor and a transmissive photointerrupter, and generates the BPF state signal B2. For example, the timing control circuit 30 monitors the BPF state signal B2 and controls a voltage or the like supplied to the servo motor through the BPF control signal B1 such that the amount of rotation displacement of the rotary shaft of the servo motor detected by the rotary encoder for a predetermined time is maintained to a predetermined amount, thereby rotating the filter rotor at a predetermined speed.

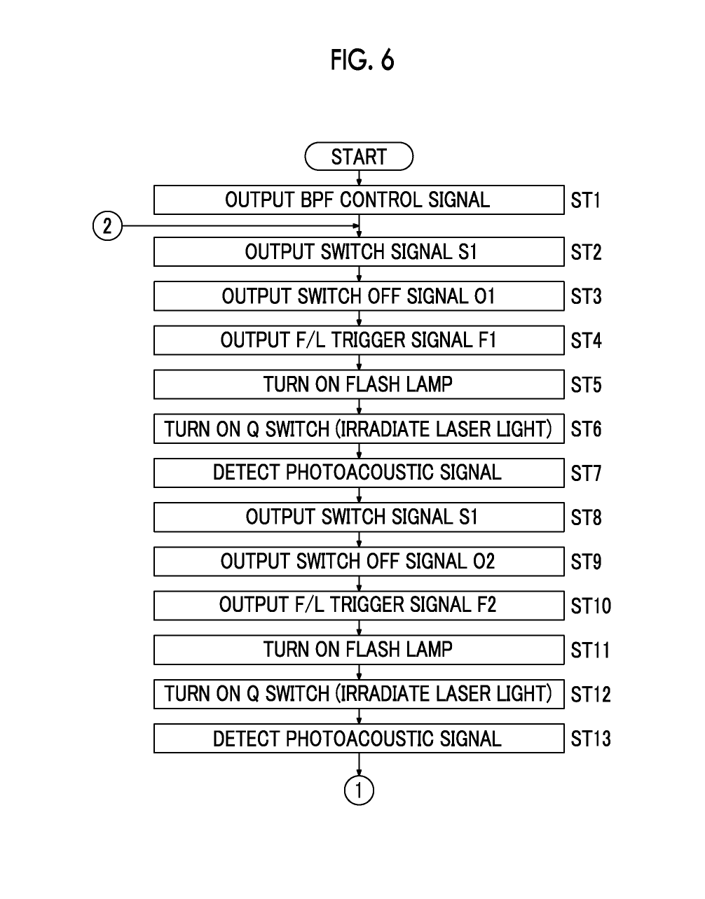

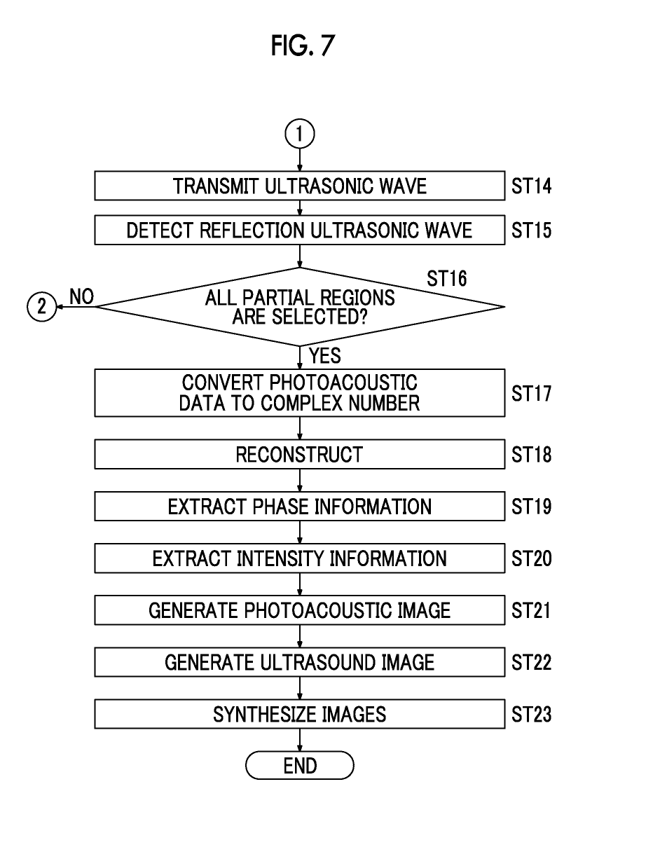

Next, the operation of the first embodiment will be described. FIGS. 6 and 7 are flowcharts showing the operation of the first embodiment. Description will be provided assuming that a region of the subject onto which laser light is irradiated is divided into a plurality of partial regions. The timing control circuit 30 outputs, to the laser light source unit 13, the BPF control signal B1 to the effect that the wavelength switching part (filter rotor) 58 in the laser light source unit 13 is rotated at a predetermined rotation speed, prior to the irradiation of pulse laser light onto the subject (Step ST1). For example, when the filter rotor shown in FIG. 5 is used and pulse laser light is emitted 24 times per second, since pulse laser light having two wavelengths of 750 nm and 800 nm during single rotation of the filter rotor can be emitted, the filter rotor may be rotated at the rotation speed of 24/2=12 rotations per second.

If the reception of the photoacoustic signal is prepared, the timing control circuit 30 outputs the switch signal S1 to the flash lamp power supply 61 of the laser light source unit 13 at a predetermined timing in order to emit pulse laser light having the first wavelength (in this case, 750 nm) (Step ST2). The switch control circuit 62 of the flash lamp power supply 61 outputs the switch signal S11 to the high pressure switch 74 to turn on the high pressure switch 74, and the high pressure switch 74 is turned on in response to the switch signal S11, whereby the charging of the capacitor 75 starts.

The timing control circuit 30 outputs the switch off signal O1 to the switch control circuit 62 when the charging time ½T0 elapses (Step ST3). The switch control circuit 62 outputs the switch off signal O11 to the high pressure switch 74 in response to the switch off signal O1, and the high pressure switch 74 is turned off in response to the switch off signal O11, whereby the charging of the capacitor 75 is completed.

The timing control circuit 30 outputs the flash lamp trigger signal F1 to the flash lamp power supply 61 when the charging of the capacitor 75 is completed (Step ST4). The trigger control circuit 63 of the flash lamp power supply 61 outputs the flash lamp trigger signal F11 to the trigger circuit 73, and the flash lamp 52 is turned on in response to the flash lamp trigger signal F11, whereby the excitation of the laser rod 51 starts (Step ST5). The timing control circuit 30 turns on the flash lamp 52 based on the BPF state signal B2, for example, at the timing calculated backward from the timing at which the rotation displacement position of the wavelength switching part 58 becomes a position where a bandpass filter which transmits light having the wavelength of 750 nm is inserted on the optical path of the optical resonator.

After the flash lamp 52 is turned on, the timing control circuit 30 turns on the Q switch 57 based on the BPF state signal B2 at the timing at which the rotation displacement position of the wavelength switching part 58 becomes a position where a bandpass filter which transmits light having the wavelength of 750 nm is inserted on the optical path of the optical resonator (Step ST6). When the Q switch 57 is turned on, since a bandpass filter having the transmission wavelength of 750 nm is inserted on the optical path of the optical resonator, the laser light source unit 13 emits pulse laser light having the wavelength of 750 nm.

For example, pulse laser light having the wavelength of 750 nm emitted from the laser light source unit 13 is guided to the probe 11 and irradiated from the probe 11 onto the first partial region of the subject. In the subject, an optical absorber absorbs energy of irradiated pulse laser light, whereby a photoacoustic signal is generated. The probe 11 detects the photoacoustic signal generated in the subject (Step ST7). The photoacoustic signal detected by the probe 11 is received by the reception circuit 21.

The timing control circuit 30 outputs the sampling trigger signal to the AD conversion part 22 in conformity with the output timing of the Q switch trigger signal Q0. The AD conversion part 22 samples the photoacoustic signal received by the reception circuit 21 in a predetermined sampling period. The photoacoustic signal sampled by the AD conversion part 22 is stored as first photoacoustic data in the reception memory 23.

Next, the control part 31 outputs the switch signal S1 to the flash lamp power supply 61 of the laser light source unit 13 at a predetermined timing in order to emit pulse laser light having the next wavelength, that is, 800 nm (Step ST8). The switch control circuit 62 of the flash lamp power supply 61 outputs the switch signal S11 to the high pressure switch 74 to turn on the high pressure switch 74, and the high pressure switch 74 is turned on in response to the switch signal S11, whereby the charging of the capacitor 75 starts.

The timing control circuit 30 outputs the switch off signal O2 to the switch control circuit 62 when the charging time T0 elapses (Step ST9). The switch control circuit 62 outputs the switch off signal O12 to the high pressure switch 74 in response to the switch off signal O2, and the high pressure switch 74 is turned off in response to the switch off signal O12, whereby the charging of the capacitor 75 is completed.

The timing control circuit 30 outputs the flash lamp trigger signal F2 to the flash lamp power supply 61 when the charging of the capacitor 75 is completed (Step ST10). The trigger control circuit 63 of the flash lamp power supply 61 outputs the flash lamp trigger signal F12 to the trigger circuit 73, and the flash lamp 52 is turned on in response to the flash lamp trigger signal F12, whereby the excitation of the laser rod 51 starts (Step ST11). The timing control circuit 30 turns on the flash lamp 52 based on the BPF state signal B2, for example, at the timing calculated backward from the timing at which the rotation displacement position of the wavelength switching part 58 becomes a position where a bandpass filter which transmits light having the wavelength of 800 nm is inserted on the optical path of the optical resonator.

After the flash lamp 52 is turned on, the timing control circuit 30 turns on the Q switch 57 based on the BPF state signal B2 at the timing at which the rotation displacement position of the wavelength switching part 58 becomes a position where a bandpass filter which transmits light having the wavelength of 800 nm is inserted on the optical path of the optical resonator (Step ST12). When the Q switch 57 is turned on, since a bandpass filter having the transmission wavelength of 800 nm is inserted on the optical path of the optical resonator, the laser light source unit 13 emits pulse laser light having the wavelength of 800 nm.

For example, pulse laser light having the wavelength of 800 nm emitted from the laser light source unit 13 is guided to the probe 11 and irradiated from the probe 11 onto the first partial region of the subject. The probe 11 detects a photoacoustic signal generated when the optical absorber in the subject absorbs pulse laser light having the wavelength of 800 nm (Step ST13). The timing control circuit 30 outputs the sampling trigger signal to the AD conversion part 22 in conformity with the output of the Q switch trigger signal Q0. The AD conversion part 22 samples the photoacoustic signal received by the reception circuit 21 in a predetermined sampling period. The photoacoustic signal sampled by the AD conversion part 22 is stored as second photoacoustic data in the reception memory 23.

Next, the control part 31 progresses the process to transmission and reception of an ultrasonic wave. The timing control circuit 30 causes an ultrasonic wave to be transmitted from the probe 11 to the subject through the transmission control circuit 37 (Step ST14). In Step ST14, the ultrasonic wave is transmitted to the same region as the partial region of the subject onto which pulse laser light is irradiated. The probe 11 detects a reflected ultrasonic wave for the transmitted ultrasonic wave (Step ST15). The detected reflected ultrasonic wave is sampled by the AD conversion part 22 through the reception circuit 21 and stored as reflected ultrasonic data in the reception memory 23.

The control part 31 performs determination about whether or not all partial regions are selected (Step ST16). When a partial region to be selected remains, the process returns to Step ST2. The photoacoustic image generation device 10 executes the processing of Steps ST2 to ST13 for the respective partial regions, sequentially irradiates pulse laser light having the respective wavelengths (750 nm and 800 nm) onto the respective partial regions, and stores the first photoacoustic data and the second photoacoustic data corresponding to the respective partial regions in the reception memory 23. Steps ST14 and ST15 are executed, and the reflected ultrasonic data is stored in the reception memory 23. If the irradiation of pulse laser light, the detection of the photoacoustic signal, and the transmission and reception of the ultrasonic wave are performed for all partial regions, data necessary for generating a photoacoustic image and an ultrasound image of one frame is gathered.

If it is determined in Step ST16 that all partial regions are selected, the control part 31 progresses the process to the generation of the photoacoustic image and the ultrasound image. The data separation part 32 separates the first and second photoacoustic data from the reflected ultrasonic data. The data separation part 32 transfers the separated first and second photoacoustic data to the complex number part 24 and transfers the reflected ultrasonic data to the ultrasound image reconstruction part 33. The complex number part 24 reads the first photoacoustic data and the second photoacoustic data from the reception memory 23, and generates complex data in which the first photoacoustic image data is a real part and the second photoacoustic image data is an imaginary part (Step ST17). The photoacoustic image reconstruction part 25 performs image reconstruction from complex data converted to a complex number in Step ST17 by a Fourier transformation method (FTA method) (Step ST18).

The phase information extraction part 26 extracts phase information from reconstructed complex data (reconstructed image) (Step ST19). For example, when reconstructed complex data is expressed by X+iY, the phase information extraction part 26 extracts θ=tan⁻¹(Y/X) as the phase information (however, when X=0, θ=90°. The intensity information extraction part 27 extracts intensity information from reconstructed complex data (Step ST20). For example, when reconstructed complex data is expressed by X+iY, the intensity information extraction part 27 extracts (X²+Y²)^{1/2} as the intensity information.

The detection and logarithmic conversion part 28 carries out the detection and logarithmic conversion processing for the intensity information extracted in Step ST20. The photoacoustic image construction part 29 generates a photoacoustic image based on the phase information extracted in Step ST19 and the intensity information extracted in Step ST20 subjected to the detection and logarithmic conversion processing (Step ST21). The photoacoustic image construction part 29 determines the luminance (gradation value) of each pixel in the distribution image of the optical absorber based on the intensity information and determines the color of each pixel based on the phase information, thereby generating a photoacoustic image.

The ultrasound image reconstruction part 33 generates data of each line of an ultrasound image by, for example, a delay addition method. The detection and logarithmic conversion part 34 obtains an envelope of data of each line output from the ultrasound image reconstruction part 33 and logarithmically converts the obtained envelope. The ultrasound image construction part 35 generates the ultrasound image based on data of each line subjected to logarithmic conversion (Step ST22). The image synthesis part 36 synthesizes the photoacoustic image and the ultrasound image, and displays the synthesized image on the image display part 14 (Step ST23).

In this way, in the first embodiment, the charging time of the capacitor 75 of the pulse generation circuit 72 which turns on the flash lamp 52 is controlled depending on the wavelength of pulse laser light to be emitted to irradiate excitation light from the flash lamp 52 onto the laser rod 51, thereafter, the oscillation wavelength is switched by the wavelength switching part 58 to adjust the wavelength of pulse laser light to be emitted, and the Q switch 57 is turned on to emit pulse laser light. Specifically, when the laser rod 51 is excited with a uniform amount of light, the charging time is set to ½T0 when emitting pulse laser light having the wavelength of 750 nm with great emission intensity from the laser light source unit 13, and, the charging time is set to T0 when emitting pulse laser light having the wavelength of 800 nm.

For this reason, when emitting pulse laser light having the wavelength of 750 nm, the amount of excitation light irradiated onto the laser rod 51 becomes ½ when emitting pulse laser light having the wavelength of 800 nm. Accordingly, it is possible to make the emission intensities of pulse laser light having the wavelength of 750 nm and pulse laser light having the wavelength of 800 nm uniform. Since the emission intensity of pulse laser light can be made uniform only by changing the charging time, the configuration of the laser light source unit 13 can be made simple and inexpensive.

As described above, since the photoacoustic image is generated by part of pulse laser light having each wavelength with uniform emission intensity, it is possible to accurately extract the magnitude relationship of relative signal intensity between photoacoustic data, and as a result, to generate a photoacoustic image suitable for diagnosis.

In this embodiment, complex data in which one of the first photoacoustic data and the second photoacoustic data obtained at two wavelengths is a real part and the other data is an imaginary part is generated, and a reconstructed image is generated from complex data by a Fourier transformation method. In this case, it is possible to efficiently perform reconstruction compared to a case where the first photoacoustic data and the second photoacoustic data are reconstructed separately. Pulse laser light having a plurality of wavelength is irradiated, and a photoacoustic signal (photoacoustic data) when irradiating pulse laser light having each wavelength is used, whereby it is possible to perform functional imaging by means of the fact that the optical absorption characteristic of each optical absorber is different depending on wavelength.

In this embodiment, for example, when a light irradiation region is divided into three partial regions, pulse laser light having the first wavelength and pulse laser light having the second wavelength are sequentially irradiated onto the first partial region, then, pulse laser light having the first wavelength and pulse laser light having the second wavelength are sequentially irradiated onto the second partial region, and thereafter, pulse laser light having the first wavelength and pulse laser light having the second wavelength are sequentially irradiated onto the third partial region. In this embodiment, after pulse laser light having the first wavelength and pulse laser light having the second wavelength are continuously irradiated onto a certain partial region, the process progresses to the next partial region. In this case, it is possible to reduce the time from when pulse laser light having the first wavelength is irradiated at the same position until pulse laser light having the second wavelength is irradiated compared to a case where pulse laser light having the first wavelength is irradiated onto the three partial regions and then pulse laser light having the second wavelength is irradiated onto the three portions. The time from when pulse laser light having the first wavelength is irradiated until pulse laser light having the second wavelength is irradiated is reduced, thereby suppressing mismatching between the first photoacoustic data and the second photoacoustic data.

In this embodiment, since the ultrasound image is generated in addition to the photoacoustic image, a portion which cannot be imaged in the photoacoustic image can be observed referring to the ultrasound image.

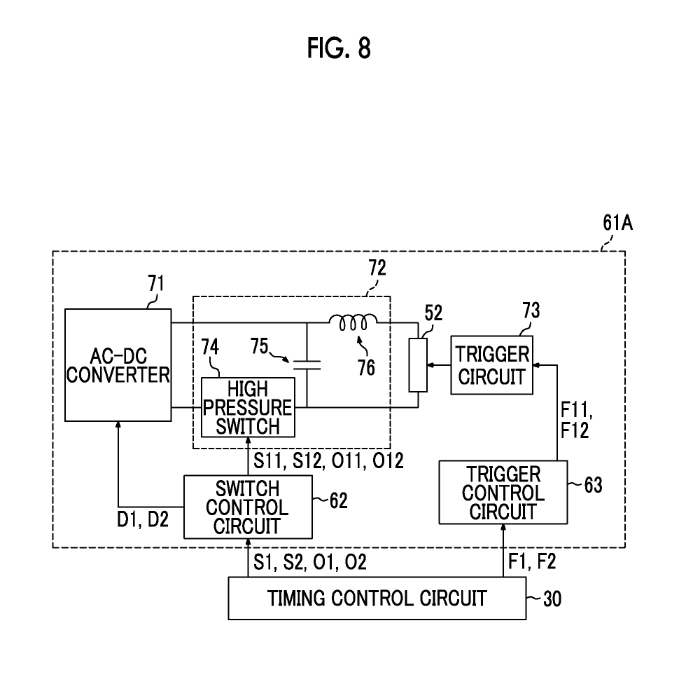

Next, a second embodiment of the invention will be described. Since the configuration of a laser light source unit according to the second embodiment and a photoacoustic image generation device to which the laser light source unit is applied is the same as in the first embodiment, here, only the configuration of a flash lamp power supply of the laser light source unit will be described. FIG. 8 is a diagram showing the configuration of the flash lamp power supply of the laser light source unit according to the second embodiment. In the second embodiment, the same configuration as in the first embodiment is represented by the same reference numerals, and detailed description thereof will be omitted. In the first embodiment, although the charging time of the capacitor 75 is controlled, the second embodiment is different from the first embodiment in that a charging voltage is controlled.

For this reason, in the second embodiment, when emitting pulse laser light having the wavelength of 750 nm, the charging voltage of the capacitor 75 is set to 1/√2 such that excitation energy becomes ½ compared to when emitting pulse laser light having the wavelength of 800 nm. Specifically, if the charging voltage when emitting pulse laser light having the wavelength of 800 nm is V0, the charging voltage of the capacitor 75 is set to 1/√2V0 when emitting pulse laser light having the wavelength of 750 nm. With this, when emitting pulse laser light having the wavelength of 750 nm, the amount of excitation light irradiated onto the laser rod 51 becomes ½ of the amount of light when emitting pulse laser light having the wavelength of 800 nm. Accordingly, it is possible to make the emission intensities of pulse laser light having the wavelength of 750 nm and pulse laser light having the wavelength of 800 nm uniform.

The charging voltages 1/√2V0 and V0 are controlled by voltage signals D1 and D2 output from the switch control circuit 62 according to the switch signals S1 and S2 output from the timing control circuit 30. That is, when emitting pulse laser light having the wavelength of 750 nm, the timing control circuit 30 outputs the switch signal S1, and the switch control circuit 62 outputs the switch signal S1 and the charging signal D1 in response to the switch signal S1. With this, the high pressure switch 74 is turned on, and the AC-DC converter 71 charges the capacitor 75 with the charging voltage 1/√2V0. When emitting pulse laser light having the wavelength of 800 nm, the timing control circuit 30 outputs the switch signal S2, and the switch control circuit 62 outputs the switch signal S12 and the charging signal D2 in response to the switch signal S2. With this, the high pressure switch 74 is turned on, and the AC-DC converter 71 charges the capacitor 75 with the charging voltage V0.

A system in which the capacitance C of the capacitor 75=100 μF, the inductance L of the coil 76=100 μH, and the pulse width=3√LC=300 μsec is considered. When the charging voltage V0 is set to 2000 V, excitation energy at the time of full charging becomes ½CV²=200 J.

If the charging time of the capacitor 75 at this time is T2, when the output P of the power supply=1500 W, T2=½CV²/P=133 msec. When excitation energy is ½, that is, 100 J, the charging voltage becomes 1414 V which is 1/√2 of 2000 V. If the charging time of the capacitor 75 at this time is T1, when the power P of the power supply=1500 W, T1=½CV×V0/P=94 msec. Accordingly, in the second embodiment, the charging time of the capacitor 75 is also controlled.

The charging time T1 and T2 are controlled by the switch off signals O1 and O2 output from the timing control circuit 30. That is, when emitting pulse laser light having the wavelength of 750 nm, the timing control circuit 30 outputs the switch off signal O1 at the timing of T1 after the charging of the capacitor 75 starts, and the switch control circuit 62 outputs the switch off signal O11 to the high pressure switch 74 in response to the switch off signal O1. With this, the high pressure switch 74 is turned off, and the charging of the capacitor 75 is completed. When emitting pulse laser light having the wavelength of 800 nm, the timing control circuit 30 outputs the switch off signal O2 at the timing of T2 after the charging of the capacitor 75 starts, and the switch control circuit 62 outputs the switch off signal O12 to the high pressure switch 74 in response to the switch off signal O2. With this, the high pressure switch 74 is turned off, and the charging of the capacitor 75 is completed.

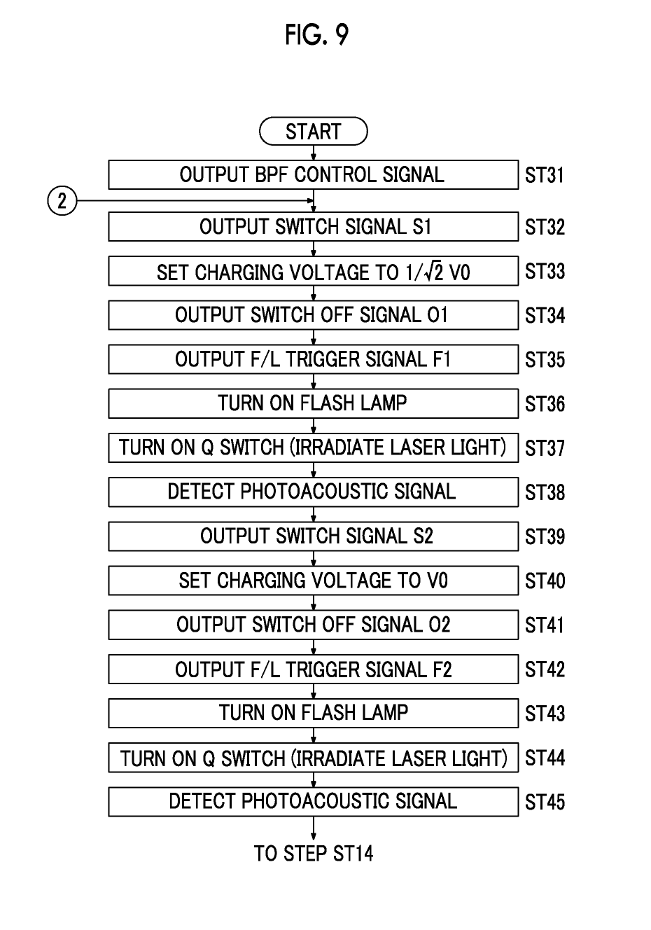

Next, the operation of the second embodiment will be described. FIG. 9 is a flowchart showing the operation of the second embodiment. As in the first embodiment, description will be provided assuming that a region of the subject onto which laser light is irradiated is divided into a plurality of partial regions. The timing control circuit 30 outputs, to the laser light source unit 13, the BPF control signal B1 to the effect that the wavelength switching part (filter rotor) 58 in the laser light source unit 13 is rotated at a predetermined rotation speed, prior to the irradiation of pulse laser light onto the subject (Step ST31).

If the reception of the photoacoustic signal is prepared, the timing control circuit 30 outputs the switch signal S1 to the flash lamp power supply 61A of the laser light source unit 13 at a predetermined timing in order to emit pulse laser light having the first wavelength (750 nm) (Step ST32). The switch control circuit 62 of the flash lamp power supply 61A outputs the charging signal D1 to the AC-DC converter 71 to set the charging voltage to 1/√2V0, and the AC-DC converter 71 sets the charging voltage to 1/√2V0 in response to the charging signal D1 (Step ST33). The switch control circuit 62 outputs the switch signal S11 to the high pressure switch 74 to turn on the high pressure switch 74, and the high pressure switch 74 is turned on in response to the switch signal S11, whereby the charging of the capacitor 75 starts.

The timing control circuit 30 outputs the switch off signal O1 to the switch control circuit 62 when the charging time T1 elapses (Step ST34). The switch control circuit 62 outputs the switch off signal O11 to the high pressure switch 74 in response to the switch off signal O1, whereby the charging of the capacitor 75 is completed.

The timing control circuit 30 outputs the flash lamp trigger signal F1 to a flash lamp power supply 61A when the charging of the capacitor 75 is completed (Step ST35). The trigger control circuit 63 of the flash lamp power supply 61A outputs the flash lamp trigger signal F11 to the trigger circuit 73, and the flash lamp 52 is turned on in response to the flash lamp trigger signal F11, whereby the excitation of the laser rod 51 starts (Step ST36). The timing control circuit 30 turns on the flash lamp 52 based on the BPF state signal B2, for example, at the timing calculated backward from the timing at which the rotation displacement position of the wavelength switching part 58 becomes a position where a bandpass filter which transmits light having the wavelength of 750 nm is inserted on the optical path of the optical resonator.

As in the first embodiment, the Q switch 57 is turned on (Step ST37), pulse laser light having the wavelength of 750 nm from the laser light source unit 13 is irradiated from the probe 11 onto the first partial region of the subject, and a thus-generated photoacoustic signal is detected (Step ST38). The detected photoacoustic signal is sampled by the AD conversion part 22 and stored as first photoacoustic data in the reception memory 23.

Next, the control part 31 outputs the switch signal S2 to the flash lamp power supply 61A of the laser light source unit 13 at a predetermined timing to emit pulse laser light having the next wavelength, that is, 800 nm (Step ST39). The switch control circuit 62 of the flash lamp power supply 61A outputs the charging signal D2 to the AC-DC converter 71 to set the charging voltage to V0, and the AC-DC converter 71 sets the charging voltage to V0 in response to the charging signal D2 (Step ST40). The switch control circuit 62 outputs the switch signal S12 to the high pressure switch 74 to turn on the high pressure switch 74, and the high pressure switch 74 is turned on in response to the switch signal S12, whereby the charging of the capacitor 75 starts.

The timing control circuit 30 outputs the switch off signal O2 to the switch control circuit 62 when the charging time T2 elapses (Step ST41). The switch control circuit 62 outputs the switch off signal O12 to the high pressure switch 74 in response to the switch off signal O2, whereby the charging of the capacitor 75 is completed.

The timing control circuit 30 outputs the flash lamp trigger signal F2 to the flash lamp power supply 61A when the charging of the capacitor 75 is completed (Step ST42). The trigger control circuit 63 of the flash lamp power supply 61A outputs the flash lamp trigger signal F12 to the trigger circuit 73, and the flash lamp 52 is turned on in response to the flash lamp trigger signal F12, whereby the excitation of the laser rod 51 starts (Step ST43). The timing control circuit 30 turns on the flash lamp 52 based on the BPF state signal B2, for example, at the timing calculated backward from the timing at which the rotation displacement position of the wavelength switching part 58 becomes a position where a bandpass filter which transmits light having the wavelength of 800 nm is inserted on the optical path of the optical resonator.

As in the first embodiment, the Q switch 57 is turned on (Step ST44), pulse laser light having the wavelength of 800 nm from the laser light source unit 13 is irradiated from the probe 11 onto the first partial region of the subject, and a thus-generated photoacoustic signal is detected (Step ST45). The detected photoacoustic signal is sampled by the AD conversion part 22 and stored as second photoacoustic data in the reception memory 23. Thereafter, the control part 31 progresses the processing of Step ST14 in FIG. 7, and generates the photoacoustic image and the ultrasound image.

In this way, in the second embodiment, the charging voltage of the capacitor 75 of the pulse generation circuit 72 which turns on the flash lamp 52 is controlled depending on the wavelength of pulse laser light to be emitted to irradiate excitation light from the flash lamp 52 onto the laser rod 51, thereafter, the oscillation wavelength is switched by the wavelength switching part 58 to adjust the wavelength of pulse laser light to be emitted, and the Q switch 57 is turned on to emit the pulse laser light. Specifically, when the laser rod 51 is excited with a uniform amount of light, the charging voltage is set to 1/√2V0 when emitting pulse laser light having the wavelength of 750 nm with great emission intensity from the laser light source unit 13, and the charging voltage is set to V0 when emitting pulse laser light having the wavelength of 800 nm.

For this reason, when emitting pulse laser light having the wavelength of 750 nm, the amount of excitation light irradiated onto the laser rod 51 becomes ½ of the amount of light when emitting pulse laser light having the wavelength of 800 nm. Accordingly, it is possible to make the emission intensity of pulse laser light having the wavelength of 750 nm and the emission intensity of pulse laser light having the wavelength of 800 nm uniform. Since the emission intensity of pulse laser light can be made uniform only by changing the charging voltage, the configuration of the laser light source unit 13 can be made simple and inexpensive.

In the above-described embodiments, although pulse laser light having two wavelengths is emitted, pulse laser light having three or more wavelengths may be emitted. In this case, excitation energy, that is, the charging time or the charging voltage may be controlled according to the emission intensity of pulse laser light at each wavelength. When generating an ultrasound image, the generation of the ultrasound image may be performed after pulse laser light having all wavelengths is irradiated onto the subject to detect a photoacoustic signal. When three or more beams of pulse laser light are used, for example, the phase information extraction part 26 may generate the magnitude relationship of relative signal intensity between the photoacoustic data corresponding to the respective wavelengths as phase information. The intensity information extraction part 27 may generate signal intensity unified from the signal intensities in the photoacoustic data corresponding to the respective wavelengths as intensity information.

In the above-described embodiments, although the wavelengths of pulse laser light are 750 nm and 800 nm, the invention is not limited thereto, and pulse laser light having various wavelengths may be emitted by changing the transmission wavelength of the bandpass filter of the wavelength switching part 58. In this case, the charging time or the charging voltage may be changed according to the wavelength to be used.

In the above-described embodiments, although the charging time or the charging voltage is changed according to the wavelength of pulse laser light to be emitted, two or more flash lamps may be provided, and the number of flash lamps to be turned on may be further changed. For example, when emitting pulse laser light having the wavelength of 750 nm, only one flash lamp may be turned on while controlling the charging time or the charging voltage, and when emitting pulse laser light having the wavelength of 800 nm, two flash lamps may be turned on while controlling the charging time or the charging voltage.

When two or more flash lamps are used, the same kind of flash lamp is not necessarily used, and different flash lamps may be combined according to the intensity ratio of the laser light output between the first wavelength and the second wavelength, the capacity of the power supply, or the like. For example, when two wavelengths at a great intensity ratio are selected, flash lamps having different impedance parameters may be combined. An impedance parameter is a parameter for determining the impedance of the flash lamp, and is determined by the light emission length, the inner diameter, and the filling pressure of gas of the flash lamp. For this reason, a flash lamp having a great inner diameter or light emission length and a flash lamp having a normal inner diameter can be combined. In this way, when flash lamps having different impedance parameters are combined, at the first wavelength (for example, 750 nm), as in the above-described embodiments, only the flash lamp having a normal inner diameter is turned on while controlling the charging time or the charging voltage, and at the second wavelength (for example, 810 nm or 815 nm, and in case of alexandrite, a wavelength at which oscillation rarely occurs), both the flash lamp having a normal inner diameter and the flash lamp having a great inner diameter are turned on while controlling the charging time or the charging voltage, whereby it is possible to make the outputs of laser light having the first wavelength and laser light having the second wavelength uniform.

In the above-described embodiments, although both the photoacoustic image and the ultrasound image are generated, only the photoacoustic image may be generated. In this case, in the ultrasound part 12, the data separation part 32, the ultrasound image reconstruction part 33, the detection and logarithmic conversion part 34, the ultrasound image construction part 35, the image synthesis part 36, and the transmission control circuit 37 can be omitted.

In the above-described embodiments, although an example where the first photoacoustic data and the second photoacoustic data are converted to a complex number has been described, the first photoacoustic data and the second photoacoustic data may be reconstructed separately without performing conversion to a complex number. Furthermore, although conversion to a complex number is performed and the ratio of the first photoacoustic data and the second photoacoustic data is computed using the phase information, the same effects are obtained even if the ratio is computed from the intensity information of both the first photoacoustic data and the second photoacoustic data. Intensity information can be generated based on signal intensity in a first reconstructed image and signal intensity in second reconstructed image.

In the above-described embodiments, although an example where the wavelength switching part 58 is configured as a filter rotor including two bandpass filter regions shown in FIG. 5 has been primarily described, the wavelength switching part 58 may switch the wavelength by rotating a birefringent filter which changes an oscillation wavelength according to rotation displacement. The invention is not limited to that using a bandpass filter or a birefringent filter, and an arbitrary configuration may be used insofar as a plurality of wavelengths can be switched.

In the above-described embodiments, although the laser light source unit is applied to the photoacoustic image generation device, the laser light source unit may be applied to an arbitrary device using pulse laser light having two or more wavelengths. The laser light source unit may be used alone.

Although the invention has been described based on the preferred embodiments, the laser light source unit and the photoacoustic image generation device of the invention are not limited to the above-described embodiments, and various corrections and alterations may be made from the configuration of the above-described embodiments and still fall within the scope of the invention.

What is claimed is:

1. A laser light source unit which sequentially emits a plurality of beams of pulse laser light in a predetermined wavelength sequence including two or more different wavelengths, the laser light source unit comprising:
a laser rod;
an excitation light source which irradiates excitation light onto the laser rod;
an optical resonator which includes a pair of mirrors facing each other with the laser rod interposed therebetween;
a laser light emission part for emitting pulse laser light;
a wavelength switching part which is inserted inside the optical resonator and changes the oscillation wavelength of the optical resonator;
an excitation light control part for controlling excitation energy supplied to the excitation light source according to the wavelength of pulse laser light to be emitted and irradiating the excitation light from the excitation light source onto the laser rod; and
an emission control part for switching the oscillation wavelength by the wavelength switching part to adjust the wavelength of pulse laser light to be emitted in synchronization with the irradiation of excitation light from the excitation light source onto the laser rod and causing the laser light emission part to emit pulse laser light.

2. The laser light source unit according to claim 1,
wherein the excitation light control part increases excitation energy as a wavelength or the emission intensity of pulse laser light from the laser rod is weak, in case when excitation is performed with a uniform amount of light.

3. The laser light source unit according to claim 1,
wherein the predetermined wavelength sequence includes a first wavelength and a second wavelength, and when the emission intensity of pulse laser light having the second wavelength is weaker than the emission intensity of pulse laser light having the first wavelength, the excitation light control part is a part for supplying first excitation energy to the excitation light source when the oscillation wavelength is the first wavelength and supplying second excitation energy greater than the first excitation energy to the excitation light source when the oscillation wavelength is the second wavelength.

4. The laser light source unit according to claim 2,
wherein the predetermined wavelength sequence includes a first wavelength and a second wavelength, and when the emission intensity of pulse laser light having the second wavelength is weaker than the emission intensity of pulse laser light having the first wavelength, the excitation light control part is a part for supplying first excitation energy to the excitation light source when the oscillation wavelength is the first wavelength and supplying second excitation energy greater than the first excitation energy to the excitation light source when the oscillation wavelength is the second wavelength.

5. The laser light source unit according to claim 3,
wherein the laser light emission part is composed of two or more flash lamps.

6. The laser light source unit according to claim 5,
wherein said two or more flash lamps differ from each other in impedance parameters.

7. The laser light source unit according to claim 3,
wherein the laser rod is alexandrite, the first wavelength is in a range of 748 nm to 770 nm, the second wavelength is in a range of 793 nm to 802 nm, and the second excitation energy has a value of 1.8 times to 2.2 times larger than the first excitation energy.

8. The laser light source unit according to claim 4,
wherein the laser rod is alexandrite, the first wavelength is in a range of 748 nm to 770 nm, the second wavelength is in a range of 793 nm to 802 nm, and the second excitation energy has a value of 1.8 times to 2.2 times larger than the first excitation energy.

9. The laser light source unit according to claim 1,
wherein the excitation light control part is a part including a capacitor, which is charged to cause the excitation light to be emitted from the excitation light source, for controlling the charging time of the capacitor according to the wavelength to control excitation energy supplied to the excitation light source.

10. The laser light source unit according to claim 9,
wherein the charging time is controlled by switch off signals.

11. The laser light source unit according to claim 9,
wherein the charging time of the first wavelength is shorter than the charging time of the second wavelength.

12. The laser light source unit according to claim 1,
wherein the excitation light control part is a part including a capacitor, which is charged to cause the excitation light to be emitted from the excitation light source, for controlling the charging voltage of the capacitor according to the wavelength to control excitation energy supplied to the excitation light source.

13. The laser light source unit according to claim 12,
wherein the charging time is controlled by switch signals.

14. The laser light source unit according to claim 12,
wherein the charging voltage of the first wavelength is smaller than the charging voltage of the second wavelength.

15. The laser light source unit according to claim 1,
wherein the laser light emission part is a Q switch which is inserted inside the optical resonator.

16. A photoacoustic image generation device comprising:
a laser light source unit according to claim 1, which sequentially emits a plurality of beams of pulse laser light in a predetermined wavelength sequence including two or more different wavelengths;
a detection part for detecting a photoacoustic signal generated in a subject when pulse laser light having each wavelength in the predetermined wavelength sequence is irradiated onto the subject and generating photoacoustic data corresponding to each wavelength based on the detected photoacoustic signal;
an intensity ratio extraction part for extracting the magnitude relationship of relative signal intensity between photoacoustic data corresponding to the respective wavelengths; and
a photoacoustic image construction part for generating a photoacoustic image based on the extracted magnitude relationship,
wherein the laser light source unit includes
a laser rod,
an excitation light source which irradiates excitation light onto the laser rod,
an optical resonator which includes a pair of mirrors facing each other with the laser rod interposed therebetween;
a laser light emission part for emitting pulse laser light,
a wavelength switching part which is inserted inside the optical resonator and changes the oscillation wavelength of the optical resonator,
an excitation light control part for controlling excitation energy supplied to the excitation light source according to the wavelength of pulse laser light to be emitted and irradiating the excitation light from the excitation light source onto the laser rod, and
an emission control part for switching the oscillation wavelength by the wavelength switching part to adjust the wavelength of pulse laser light to be emitted in synchronization with the irradiation of excitation light from the excitation light source onto the laser rod and causing the laser light emission part to emit pulse laser light.

17. The photoacoustic image generation device according to claim 16, further comprising:
an intensity information extraction part for generating intensity information representing signal intensity based on photoacoustic data corresponding to each wavelength,
wherein the photoacoustic image construction part is a part for determining the gradation value of each pixel of the photoacoustic image based on the intensity information and determining the display color of each pixel based on the extracted magnitude relationship.

18. The photoacoustic image generation device according to claim 16,
wherein the detection part is a part for detecting a reflected ultrasonic wave to an ultrasonic wave transmitted to the subject to generate reflected ultrasonic data, and
the photoacoustic image generation device further includes ultrasound image generation part for generating an ultrasound image based on the reflected ultrasonic data.

19. A method of controlling a laser light source unit according to claim 1, which sequentially emits a plurality of beams of pulse laser light in a predetermined wavelength sequence including two or more different wavelengths, the method comprising:
a step of controlling excitation energy supplied to an excitation light source, which irradiates excitation light onto a laser rod, among two or more excitation light sources, which irradiate excitation light onto the laser rod, according to the wavelength of pulse laser light to be emitted;
a step of irradiating excitation light from the controlled excitation light source onto the laser rod;
a step of switching an oscillation wavelength by a wavelength switching part, which is inserted inside an optical resonator including a pair of mirrors facing each other with the laser rod interposed therebetween and changes the oscillation wavelength of the optical resonator, to adjust the wavelength of pulse laser light to be emitted in synchronization with the irradiation of excitation light from the excitation light source onto the laser rod; and
a step of causing a laser light emission part for emitting pulse laser light to emit pulse laser light.
